(19) United States
(12) Patent Application Publication
(10) Pub. No.: US 2014/0221891 A1
(43) Pub. Date: Aug. 7, 2014

(54) Brace For Unloading Of Medial Or Lateral Joint

(71) Applicant: Matrix Bracing, Inc., Irvine, CA (US)

(72) Inventors: Murali Sreeramagiri, Irvine, CA (US); Samuel Jackson Rhodes, Los Angeles, CA (US); William R. Smith, Long Beach, CA (US)

(73) Assignee: Matrix Bracing, Inc., Irvine, CA (US)

(21) Appl. No.: 14/171,621

(22) Filed: Feb. 3, 2014

Related U.S. Application Data
(60) Provisional application No. 61/760,475, filed on Feb. 4, 2013.
(60) Provisional application No. 61/760,455, filed on Feb. 4, 2013.

(51) Int. Cl. A61F 5/01

(52) U.S. Cl. CPC A61F 5/0123
USPC 602/16

(57) ABSTRACT

A knee brace for easing the symptoms of osteoarthritis is disclosed in which force straps provide a varying unloading force to the knee as the joint is flexed and straightened. The force straps are arranged to alter the degree of angulation between frames of the brace that span the joint, such that abstracting force is applied above and below the joint rather than to the joint itself. A dual hinge brace incorporating a push-pull hinge that alters the degree of angulation between the frames of a knee brace is also described.

This application claims priority to U.S. Provisional Application 61/760,475, filed Feb. 4, 2013, and U.S. Provisional Application 61/760,455, filed Feb. 4, 2013. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is orthotic devices and orthopedic braces, particularly for application to the knee.

### BACKGROUND

Osteoarthritis, a condition that results in joint inflammation, is a growing problem in an aging and increasingly obese population. It is particularly problematic when it occurs in weight bearing joints, such as the knee, resulting in pain, reduced mobility, and progressive disability in affected individuals. Osteoarthritis of the knee results in the loss of the smooth cartilage lining of the knee. This reduces the joint space within the knee, and leads to damage of the ends of the bone within the joint. The reduced space results in bone coming directly into contact with bone, and the resulting abnormal distribution of pressure across the knee joint can result in the formation of painful bone spurs. The condition may be treated using a variety of modalities, including the use anti-inflammatory drugs, surgery to remove diseased tissue, and the implantation of artificial joints or joint surfaces. Affected individuals may find symptomatic relief from osteoarthritis through the use of devices such as shoe wedges, canes, and orthopedic braces that support and relieve pressure on affected joints. For example, an individual with a knee affected by osteoarthritis may find relief by using a brace that functions by reducing the load on the knee.

One approach to reducing the load on an arthritic knee is to use a brace that provides support above and below the knee joint using a pair of cuffs or frames. A variety of approaches to providing adequate and appropriate support have been developed. Some braces, such those described in U.S. Pat. No. 3,902,482 (to Taylor) assume part of the overall load applied to the knee. The described device utilizes a robust and complex hinge mechanism that supports weight while mimicking the complex natural movements of the knee. While this approach can aid in supporting the damaged joint, the mechanism itself can be heavy and is subject to significant friction and wear.

An alternative approach exploits the fact that osteoarthritis frequently develops in only a portion of the knee joint. In such cases considerable relief from osteoarthritis symptoms can be obtained by applying lateral or medial force, effectively “unloading” mechanical stress on the diseased and damaged tissue. Such unloading braces function (at least in part) by providing abstraction, or separation, of affected joint surface.

Some unloading knee braces apply such lateral or medial force utilizing a hinge mechanism, where the hinge joins cuffs on either side of the knee. For example, U.S. Pat. No. 5,400,806 discloses an unloading brace that includes an adjustable hinge. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

The hinge is adjusted by a physician to apply lateral or medial force to the knee joint that provides abstraction of the joint on the side opposite that of the hinge. Such a brace, however, supplies this force on a constant basis, whereas the application of such force is only necessary when the knee is bearing weight. In addition, such a brace limits the normal movements of the knee. United States Patent No. U.S. Pat. No. 8,292,838 (to Ingimundarson and Kimes) disclose a brace that includes a pair of hinges that allow the device to more closely mimic the mobility of the knee, but at the expense of additional weight, friction, and complexity.

Other unloading braces rely on straps for the application of abstracting force. For example, U.S. Pat. No. 5,277,698 (to Taylor) describes an unloading brace that includes a relatively simple hinge and a single strap. This strap winds helically around the knee, such that extension of the leg tightens the strap and forces it against one side the knee joint. This urges the knee joint towards the hinge, widening the space within the joint on the side of the knee adjacent to the hinge. Similarly, United States Patent No. U.S. Pat. No. 7,198,610 (to Ingimundarson and Einarsson), U.S. Pat. No. 7,713,225 (to Ingimundarson and Einarsson), and U.S. Pat. No. 7,794,418 (to Ingimundarson and Einarsson) disclose unloading braces that utilizes a pair of straps (or a single, folded long strap) that are affixed to the cuffs of the brace so that they press against the knee joint as the leg is extended.

In order to be safe and effective, however, such braces require the use of a robust hinge mechanism positioned at a significant distance from the knee joint, in order to prevent direct contact between the knee and the hinge mechanism. Such contact would result in forces being applied to the knee by the hinge that oppose those applied by the strap, reducing the effectiveness of the brace. In addition, the amount of force that can be generated by such designs has inherent limitations. For example, flexibility of the straps limits the amount of force they can apply to the joint. While this may be partially compensated for by increasing the tension on the straps or the use of highly inelastic material, such approaches can make the appliance difficult to apply and subject to premature wear. As a result such braces tend to be heavy, bulky, and complex, making convenient application by an individual with osteoarthritis problematic at best. In addition the inherent limitations in the amount of force that such devices can apply may limit their utility for users with more advanced disease.

Thus, there is still a need for a simple and effective orthopedic brace for relief of osteoarthritic joints.

### SUMMARY OF THE INVENTION

The inventive subject matter provides apparatus and methods in which a brace is used to provide an unloading force to a joint. The brace includes upper and lower frames that are joined by a hinge and at least one force strap. The force strap is arranged to provide a force that changes the angle between the upper and lower frames as the limb is extended. This changed angle causes the upper and lower frames to apply pressure (via the upper and lower frames) to the limb both above and below the joint to which the brace is applied, for example a knee affected by osteoarthritis. The resulting pressure is directed towards the hinge, and generates an unloading force that is applied to the affected joint. This unloading force abstracts the joint and in doing so increases compartmental space on the side of the joint away from the hinge. This increase in compartmental space alleviates at least some of the symptoms associated with osteoarthritis.

One embodiment of the inventive concept is a brace for applying an unloading force to a joint, where a brace (which is configured to be applied to a limb) includes a first frame having a first aspect (for example, a medial aspect) and a second aspect (for example a lateral aspect), a second frame having a similar first aspect and a second aspect, a hinge that lies between the first frame and the second frame, and a force strap. Such frames are preferably constructed of a material that is at least partially pliant (for example polyamide, polyurethane, polypropylene, crosslinked polypropylene, polyethylene, crosslinked polyethylene, carbon fiber, fiberglass, resin, or a combination of these). In some embodiments the hinge is configured to confer an angulation to the brace, and in still other embodiments this angulation changes as the limb to which the brace is applied straightens and bends. The force strap has a first terminus, a second terminus, and an intermediate segment that lies between the first terminus and the second terminus. In the assembled brace the first terminus of the force strap is connected to the first aspect of the first frame (for example, via a swivel connector), the intermediate segment crosses over to the second aspect of the first frame, and is then directed to to the first aspect of the second frame, where second terminus is attached. The intermediate segment strap is engaged with the second aspect of the first frame in a manner that permits it to slide. This arrangement causes the angle between the first frame and the second frame to increase along a first axis as the limb to which the brace is applied is extended along a second axis. In a preferred embodiment of the inventive concept the first axis is normal to the second axis.

In some embodiments of the inventive concept the hinge can be configured to cause the angle between the first frame and the second frame to increase along a first axis as the limb to which the brace is applied is extended along a second axis. In such embodiments the first axis can be normal to the second axis. Such a hinge can be adjustable, for example by a user. The hinge can include a ramping mechanism that applies graduated pressure as the limb extends and rotates the hinge that facilitates this angulation. In an alternative embodiment, the hinge includes a pad that is mechanically coupled to a ramping mechanism such that it also moves along this first axis as the limb extends along the second axis.

In some embodiments the brace includes a second force strap that is arranged in a manner similar to that of the first force strap, but in a reversed orientation. In such an embodiment the second force strap includes a third terminus, a fourth terminus, and a second intermediate segment that lies between the third terminus and the fourth terminus. This third terminus is attached to the second aspect of the second frame, with the second intermediate segment crossing over to the first aspect of the second frame and then directed to the first frame, where the fourth terminus is attached to the second aspect of the first frame. The intermediate segment of the second force strap is engaged with the first aspect of the second frame in a manner that permits it to slide.

In an alternative embodiment, the brace includes circumferential straps associated with the first and second frames, which assist in securing the position of the brace. Such circumferential straps can be elastic or can be inelastic, whereas force straps are preferably inelastic. The lengths of the force straps and/or the circumferential straps is adjustable, and such straps can include indicia (i.e. stripes, numbers, letters, etc.) that permit a user to easily set the straps to a desired or predetermined length.

Another embodiment of the inventive concept is a method for providing an unloading force to a joint, in which a brace that includes a first frame having a first aspect and a second aspect, a second frame having a first aspect and a second aspect, and a force strap that has a first terminus, a second terminus, and an intermediate segment that lies between the first terminus and the second terminus is provided. The force strap of the brace is arranged such that the first terminus is attached to the first aspect of the first frame and the second terminus is attached to the first aspect of the second frame, and the intermediate segment strap is engaged with the second aspect of the first frame in a manner that permits it to slide. This brace is applied to a limb that has a joint, such that the joint is placed between the first frame and the second frame. In such an embodiment the angulation between the first frame and the second frame increases when the limb is extended. In some embodiments the supplied brace includes a second force strap, where second force strap includes a third terminus, a fourth terminus, and a second intermediate segment that lies between the third terminus and the fourth terminus. In such an embodiment the third terminus is affixed to the second aspect of the second frame and the fourth terminus is affixed to the second aspect of the first frame, with the second intermediate segment engaged with the first aspect of the second frame in a manner that permits it to slide. In some embodiments of the inventive method the angulation between the first frame and the second frame that occurs as the limb is extended is modified by adjusting the length of the first force strap, the second force strap, or both force straps.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A shows an orthogonal view of an embodiment of a brace of the inventive concept.

FIG. 1B shows an anterior-to-posterior view of an embodiment of a brace of the inventive concept.

FIG. 1C shows a posterior-to-anterior view of an embodiment of a brace of the inventive concept.

FIG. 1D shows a medial-to-lateral view of an embodiment of a brace of the inventive concept.

FIG. 1E shows a lateral-to-medial view of an embodiment of a brace of the inventive concept.

FIG. 2 depicts an embodiment of a brace of the inventive concept, showing pads associated with the frames of the brace.

FIG. 3A shows an anterior view of a knee joint affected by osteoarthritis.

FIG. 3B shows an orthogonal view of a flexed knee joint affected by osteoarthritis, in which the individual is wearing a brace of the inventive concept.

FIG. 3C shows an anterior view of an extended knee joint affected by osteoarthritis, in which the individual is wearing a brace of the inventive concept. Large arrows depict forces applied by the frames of the brace when the leg is in an extended position.

FIG. 4A depicts a portion of a mushroom catch strap fixator.

FIG. 4B depicts a mushroom cap strap fixator in use.

FIG. 5A depicts a male portion of a snap strap fixator.

FIG. 5B depicts a female portion of a snap strap fixator.

FIG. 5C depicts a snap strap fixator in use.

FIG. 6 shows an embodiment of a hinge of the inventive concept in an exploded view.

FIG. 7A shows an embodiment of a ramping hinge of the inventive concept in an exploded view.

FIG. 7B shows an embodiment of a ramping hinge of the inventive concept with the ramping hinge in the straight position, with covering components omitted to illustrate the internal mechanism.

FIG. 7C shows an embodiment of a ramping hinge of the inventive concept with the ramping hinge in a flexed position, with covering components omitted to illustrate the internal mechanism.

FIG. 8A depicts an alternative embodiment of a brace of the inventive concept, having a push-pull hinge and a internal center of rotation (ICR) hinge.

FIG. 8B depicts an embodiment of a push-pull hinge for use with a brace of the inventive concept.

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

One should appreciate that the devices and methods described herein advantageously permit the abstraction of a joint (for example, a knee afflicted with osteoarthritis) without the application of pressure directly to the affected joint, which greatly improves the comfort of the user, and does so using a simple and robust mechanism that is straightforward to don and considerably less bulky and heavy than prior art designs. The design of the brace also facilitates application and adjustment while in a seated position, which is particularly advantageous for elderly users whose balance may be impaired.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Similarly, it should be appreciated that, examples described and depicted in figures herein may show that are adapted for use on a limb on a specific side of the body or that are adapted for use in the support of a joint that is affected on a specific side. It should be understood that embodiments of the inventive concept include embodiments in which the arrangement of components and mechanisms described herein is reflected, reoriented, or otherwise adapted in order to permit utilization on either side of the body and with joints affected in different areas.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

One embodiment of the inventive concept is a brace for use in providing unloading of a joint, for example a knee, that is affected by osteoarthritis. In many cases osteoarthritis affects one side of the joint to a greater extent than the other. When this occurs, considerable relief can be afforded to the affected individual by the utilization of a brace that spans the affected joint and provides a force that causes abstraction or opening of the space within the joint on the affected side. This reduces friction across damaged and frequently inflamed tissue within the joint on the affected side, greatly improving comfort and mobility. In the case of an individual with a knee that is so affected it is particularly important that an effective amount of force be applied when the leg/knee joint is extended and in weight-bearing position. It is also particularly advantageous to at least partially relieve the abstractive force when the leg or knee joint is flexed, as in this position the joint is not weight bearing and the abstractive force, which can be uncomfortable and/or inconvenient to the wearer of the brace, is not as necessary.

One embodiment of the inventive concept is single hinge osteoarthritis (OA) brace that utilizes a novel and highly effective force strap system to create an unloading force on either the Medial (Varus unloading) or Lateral knee joint (Valgus unloading), depending upon which side of the brace the hinge and straps are located, by changing the angle between a pair of frames that span the affected joint as the flexion of the knee changes. In such embodiments force straps are arranged in a “Z” pattern that provides unloading force as the knee is extended without contacting the knee joint directly. The force that is applied is a function of the points at which the force straps interact with frames of such a brace and the length of the force straps. This greatly improves the wearer's comfort compared to prior art designs, in which force straps press directly against the affected joint when in use. A brace of the inventive concept can include a single force strap, two force straps, three force straps, or four or more force straps. In a preferred embodiment of the inventive concept the brace includes two force straps. A brace of the inventive concept can also include one or more stabilizing or anterior straps, which partially encircle the limb when the brace is in use and aid in stabilizing its position.

Straps utilized on a brace of the inventive concept (particularly force straps) can be essentially inelastic, and constructed of any suitable material that can permit free movement of the strap through portions the brace and the application of sufficient force for joint abstraction. Suitable materials for straps include leather, plastic, nylon (polyamide), acrylic, polyester, acetate, cotton, hemp, or a combination of these. Straps can also include indicia that can be used to adjust the length of a strap to a prescribed or desired length. Such indicia include printed stripes, raised areas (for example, stitching running perpendicular to the length of the strap), letters, numbers, or other symbols suitable for visual or tactile identification by a user. It should be appreciated that such indicia are useful to a physician, orthotist, physical therapist, and/or other medical professionals in setting the brace to apply a prescribed amount of abstracting force. Similarly, such indicia are useful to a brace wearer who may wish to alter or adjust the amount of abstracting force that is applied for comfort or to adapt for different conditions. Alternatively, a strap can be supplied with perforations and a buckle mechanism that permits length adjustment. In still other embodiments, a strap can include a hook-loop closure that permits length adjustment. Force straps and other straps utilized in a brace of the inventive concept can terminate in devices or connectors that permit easy attachment of the strap to a portion of the brace and permit the force strap to swivel or rotate through a plane parallel to an aspect of the brace, such as a snap catch or a mushroom catch (described below). In some embodiments of the inventive concept, force straps are constructed of essentially inelastic materials whereas stabilizing straps are constructed of elastic or partially elastic materials.

As noted above, the abstracting force of a brace of the inventive concept is generated by the force straps and applied to the limb by a pair of frames that span the affected joint. In some embodiments these frames are constructed of an at least partially pliant material, which reduces pressure to the limb as the abstracting force is applied and improves wearer comfort. It should also be appreciated that such materials simplify donning and doffing of the brace, and thereby improve compliance. Suitable materials include polyamide, polyurethane, polypropylene, crosslinked polypropylene, polyethylene, crosslinked polyethylene, carbon fiber, fiberglass, resin, or a combination of these. The thickness of such materials can be selected to give the desired combination of support and flexibility. In some embodiments, the material of the frame can be from 1 mm to 10 mm thick. In other embodiments, the material of the frame can be 2 mm to 8 mm thick. In still other embodiments, the materials of the frame can be approximately 5 mm in thickness. It should be appreciated, however, that the thickness of the frame materials may vary in different locations of the frame, and can be thicker in areas experiencing greater stress (for example, at or near force strap attachment and inflection points). In a preferred embodiment of the inventive concept, a frame of the brace is made from nylon (polyamide) of a thickness that provides support while remaining pliant enough to simplify donning and doffing of the appliance. Frames of the inventive concept can be produced by injection molding, which can be customized to the dimensions of an individual wearer. Alternatively, frames can be produced in fixed sizes that are then adapted to individual wearers by methods known in the art. In still other embodiments, frames can be produced by adapting a sheet of frame material to an individual wearer, for example by an orthotist using a thermal plastic material to form at least a portion of a frame.

Frames of the inventive concept can include vents or apertures. These can serve to reduce weight, and to serve as anchor points or inflection points for straps. For example, a strap can be passed through an aperture and looped back upon itself to affix a terminus of a strap to a frame, thereby serving as an anchor point. Alternatively, a strap can pass through an aperture of a frame, bend or inflect, and be redirected to another location of the brace where it can be anchored.

The frames of a brace of the inventive concept can be joined by one or more hinges. Such a hinge be a simple hinge, for example a hinge that mimics or reproduces the motion of the knee joint. Alternatively, such a hinge can be a ramping hinge, which can apply varying amounts of abstracting force or pressure as the hinge moves. Hinges can include a pad that protects the wearer from the moving components of the hinge and reduces pressure on the joint should the hinge or portions thereof come into contact.

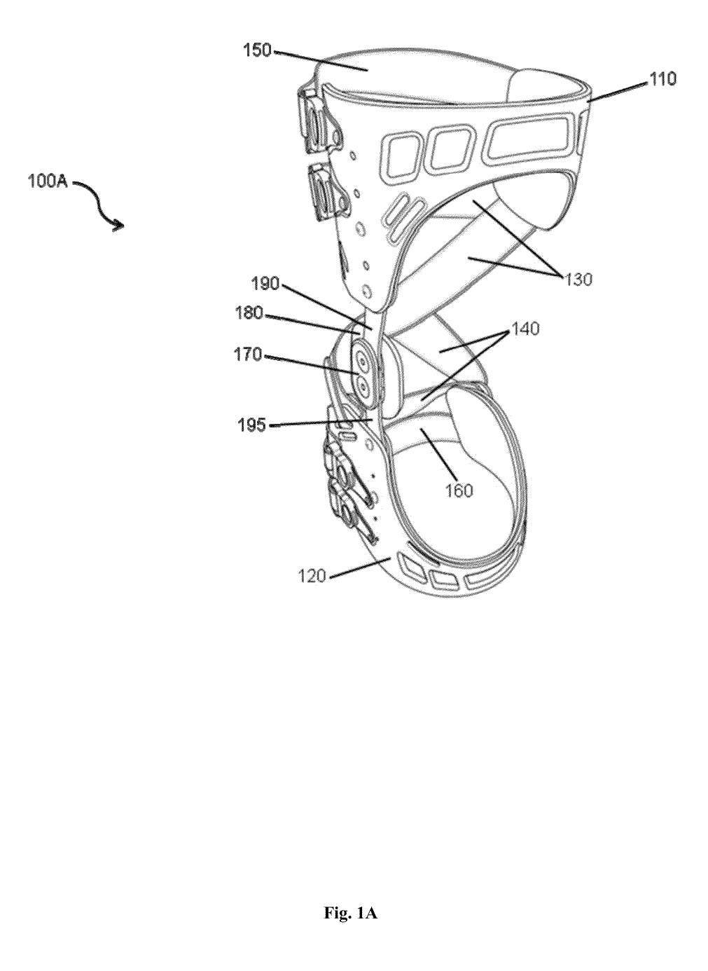

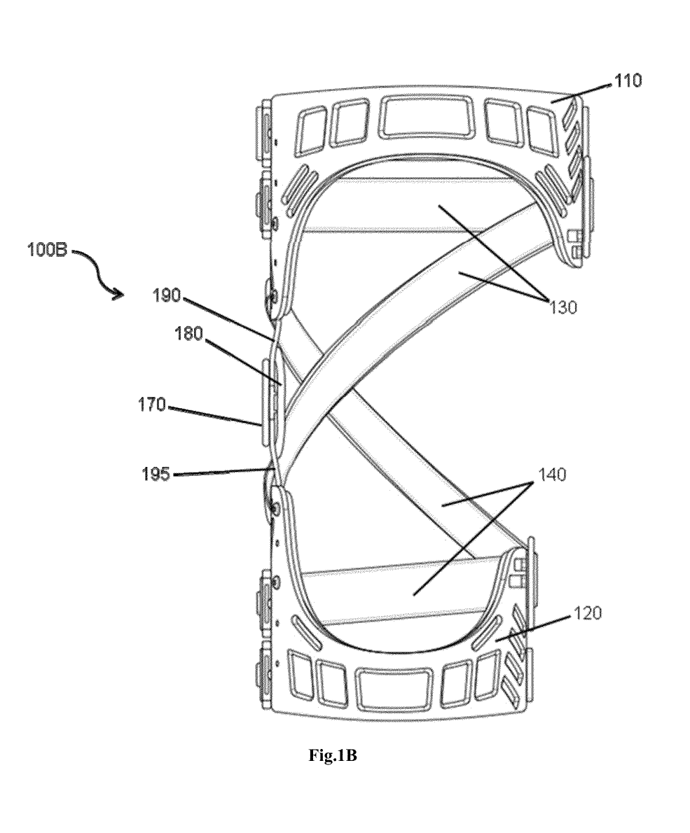

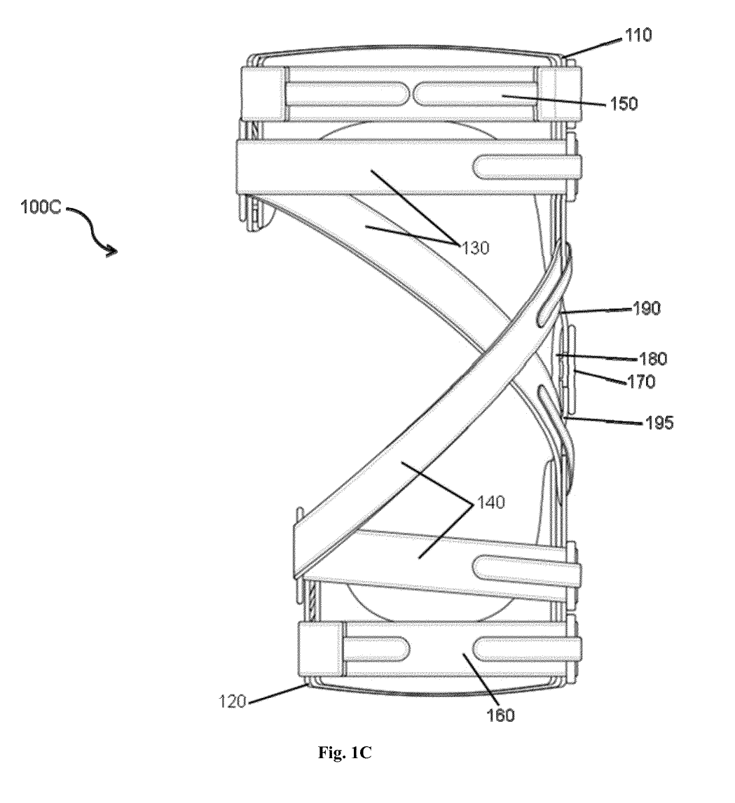

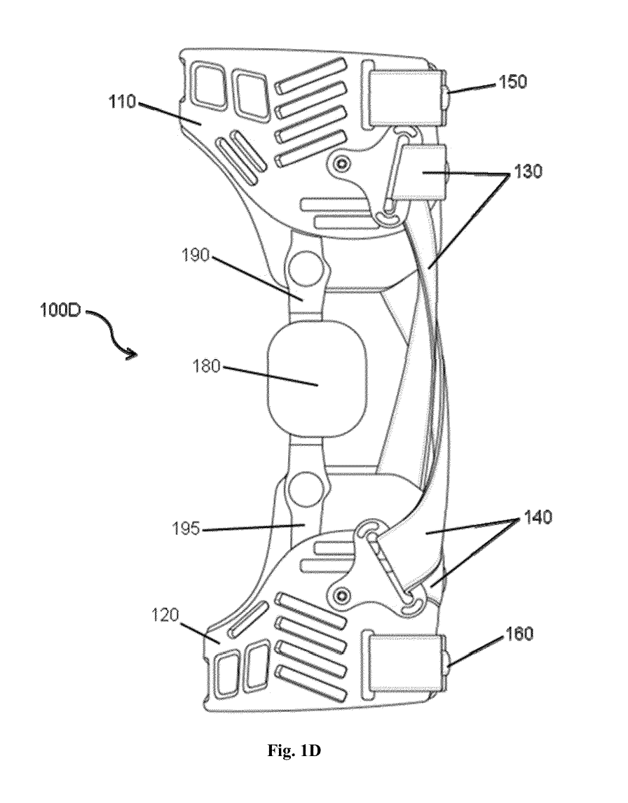

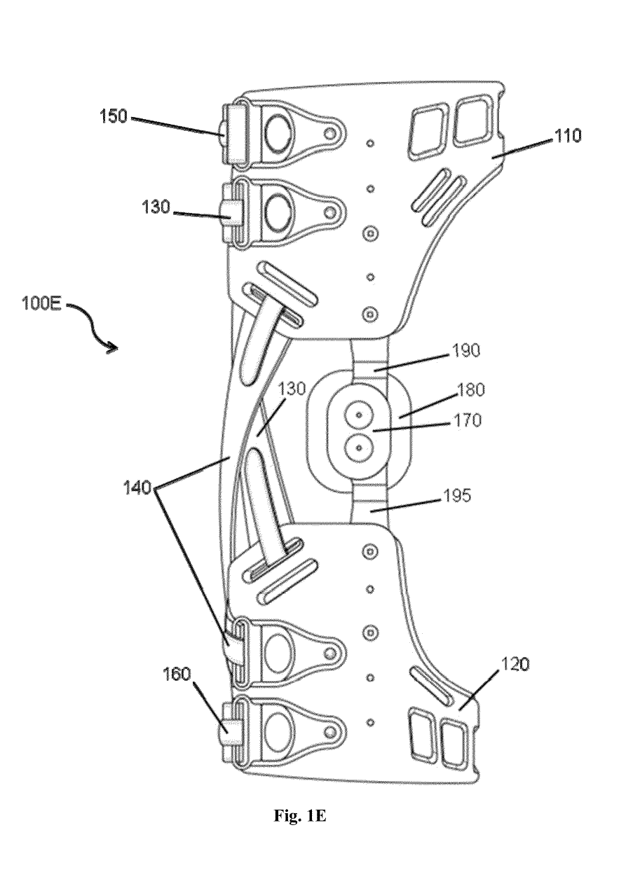

An example of a knee brace of the inventive concept is depicted in FIGS. 1A to 1E. FIG. 1A depicts an orthogonal view of a knee brace of the inventive concept 100A. The brace includes an upper frame 110 and a lower frame 120 that span the knee joint. An upper force strap 130 is attached at one side of the upper frame 110 (the left side in this depiction), crosses over to the opposite side of the upper frame, and turns (for example, by passing through a slot or other aperture through which it can slide freely) to attach to the lower frame 120 on the same side that it is attached to the upper frame 110. A lower force strap 140 follows a similar, but inverted path, with connections to the lower frame 120 and upper frame. This arrangement forms a “Z” pattern that effectively draws the force straps 130, 140 tight as the leg is extended and can be seen in FIGS. 1B and 1C, which depict anterior-to-posterior 100B and posterior-to-anterior 100C views, respectively. The brace can also include anterior or stabilizing straps 150, 160 associated with the frames 110, 120 that aid in positioning and securing the brace to the leg and that reduce rotation of the brace during use.

The tightening of the force straps 130, 140 changes the relative angle between the upper frame 110 and the lower frame 120, impelling them against the wearer's leg above and below the affected knee. This generates an unloading force that is at least partially directed towards a hinge 170 that is connected to the upper frame 110 via an upper arm 190 and to the lower frame 120 via a lower arm 195. It should be appreciated that the unloading or abstractive force applied by the brace can be modified by adjusting the length of one or both of the arms 190, 195. Alternatively, the unloading or abstractive force applied by the brace can be modified by adjusting the position along an arm 190, 195 at which it is affixed to a frame of the brace. Towards that end an arm 190, 195 can include two or more attachment sites (for example, apertures that admit a connector projecting from a frame), where such attachment sites are configured to affix the arm to a frame of the brace. The hinge 170 can also include a pad 180, which protects the wearer from the moving components of the hinge and improves wearer comfort should components of the hinge 170 contact the wearer. The pad 180 and its relationship with the hinge 170 can be seen in FIG. 1D and 1E, which depict medial-to-lateral 100D and lateral-to-medial views, respectively, of a brace of the inventive concept.

In some embodiments the hinge 170 can be constructed to provide a fixed angle between the upper frame 110 and the lower frame 120, so as to apply an abstractive pressure in addition to that supplied by the force straps 130, 140. In other embodiments the hinge 170 is constructed to provide a changing angle between the upper frame 110 and the lower frame 120 as the knee is flexed and straightened, so as to apply increasing abstracting pressure as the leg is extended.

In some embodiments of the inventive concept, the hinge 170 can be configured such that the pad 180 is pressed against the unaffected side of the knee. In such embodiments the pad 180 can act as a fulcrum, providing further abstracting pressure upon the joint, which can be useful in the treatment of individuals with more advanced disease. In an alternative embodiment of the inventive concept, the hinge 170 can be configured such that the pad 180 applies pressure to the adjacent joint surface that increases as the leg is extended (i.e. a ramping hinge), for example by movement of the pad 180 towards the knee as the knee is straightened and away from the knee as the knee is flexed.

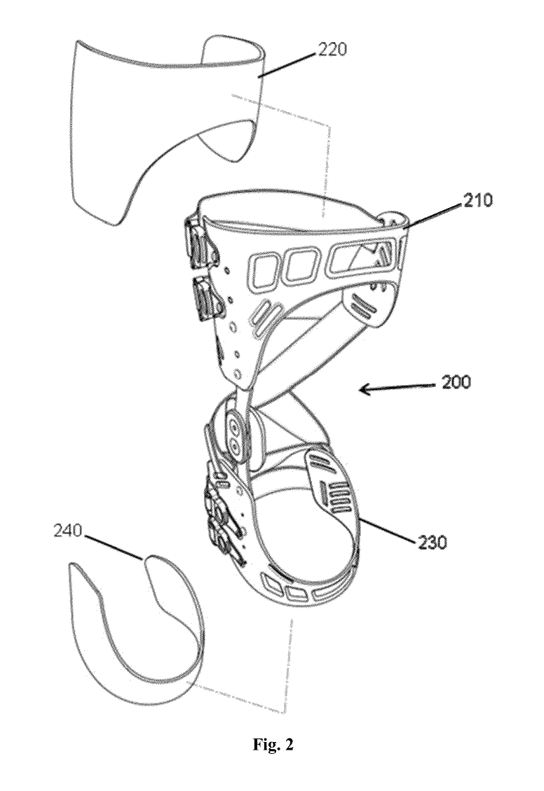

Embodiments of the inventive concept can include a pad or liner that at least partially cover an interior surface of a frame of the brace. Such a pad or liner can serve to increase the comfort of the wearer and/or to increase the efficiency of the brace by helping to hold it in place. FIG. 2 shows a brace 200 (similar to that depicted in FIG. 1A), with an upper frame 210 and an associated upper frame pad 220, along with a lower frame 230 and an associated lower frame pad 230. Pads can be constructed from any suitable materials, for example textiles, rubber foam, silicone, etc. It should be appreciated that such upper frame pads 220 and lower frame pads 240 need not be constructed of the same materials. In a preferred embodiment, the lower frame pad 240 is constructed of a material that provides a degree of traction or adhesion to the skin surface (for example, polyurethane, silicone, etc.) in order to aid in positioning and fixation of the brace, while the upper frame pad 220 is constructed of a different material provides padding (for example, fabric, plastic foam, etc.). Such pads can be integrated into the frames during manufacturing or, alternatively, be attached to the frames in a removable manner so as to facilitate cleaning and replacement.

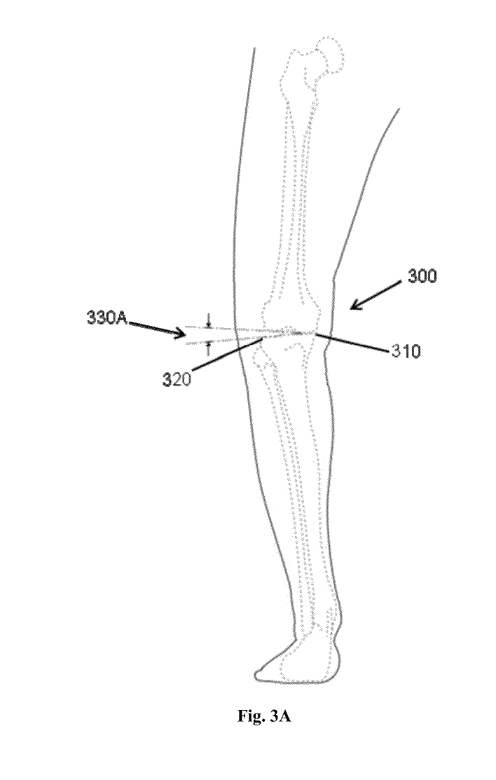

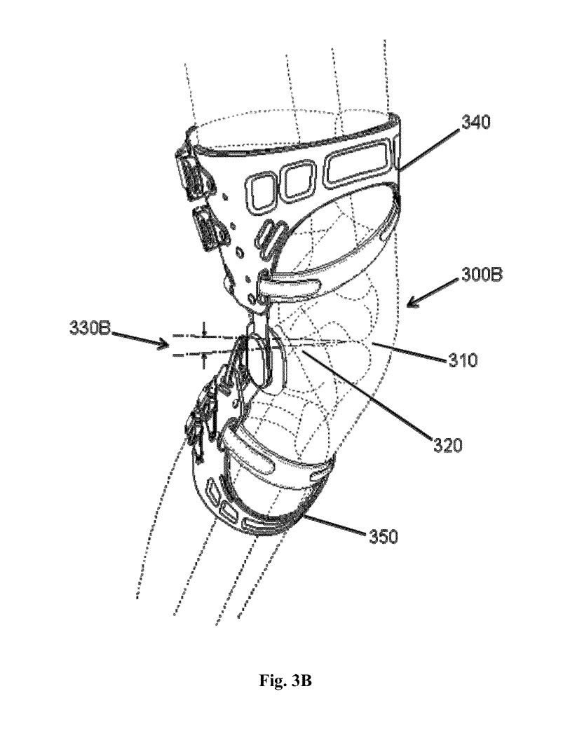

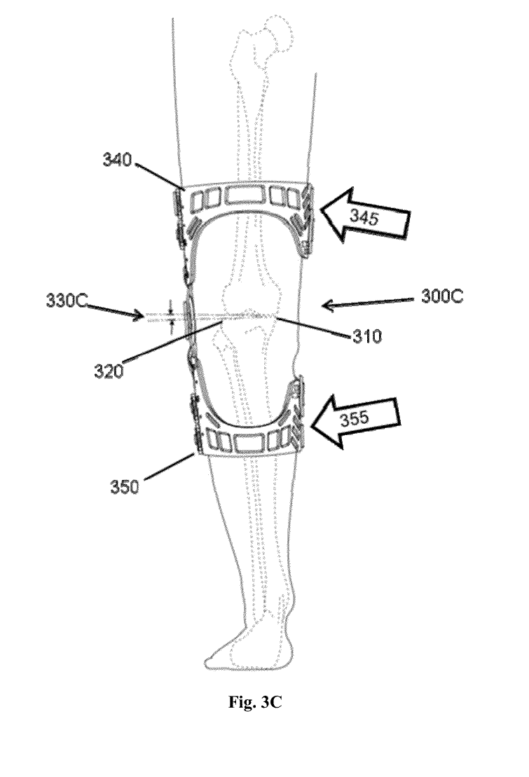

FIGS. 3A to 3C depict the utilization of a brace of the inventive concept in treatment of an individual having osteoarthritis of the knee. FIG. 3A depicts a view of the anterior of the right leg of an individual with a typical case of osteoarthritis of the knee 300. The positions of the bones of the leg are shown in broken lines. In this instance (and as is frequently the case), the medial side of the joint 310 is affected, with loss of cartilage leading to a loss of volume in the joint on this side. This results in painful contact between the bones of the upper and lower leg when the leg is straightened and weight bearing. The increased lateral angle 330A of the joint also increases joint volume on the unaffected lateral side 320.

FIG. 3B shows the individual of FIG. 3A with a brace of the inventive concept while in a seated position. The affected knee 300B is bounded by the upper frame 340 and the lower frame 350 of the brace, however since the knee is flexed the brace does not exert abstracting pressure. As a result the affected side of the knee 310 continues to relatively low volume relative to the unaffected side of the joint 320, and the angle of the knee 330B remains relatively unaffected. In the absence of weight bearing, however, contact between the bones of the upper and lower leg is avoided and the abstracting pressure is not necessary. In addition to avoiding possible discomfort to the patient by the application of pressure when it is not needed, this lack of abstracting pressure while the knee is flexed (for example, when the patient is seated) greatly simplifies application of the brace and improves compliance.

FIG. 3C depicts an individual as in FIG. 3B with the braced knee straightened/leg extended. This changes the angle between the upper frame 340 and the lower frame 350, which in turn applies abstractive pressure above 345 and below 355 the affected knee 300C. This results in abstraction of the affected side of the joint 310, increasing volume within the joint space at this location and reducing friction and pain within the joint. This is accompanied by a return to a more normal joint space on the unaffected side 320. The action of the brace also decreases the angle 330C of the joint. It should be appreciated that this is accomplished without applying pressure directly to the joint itself, which is problematic in an arthritic and painful knee.

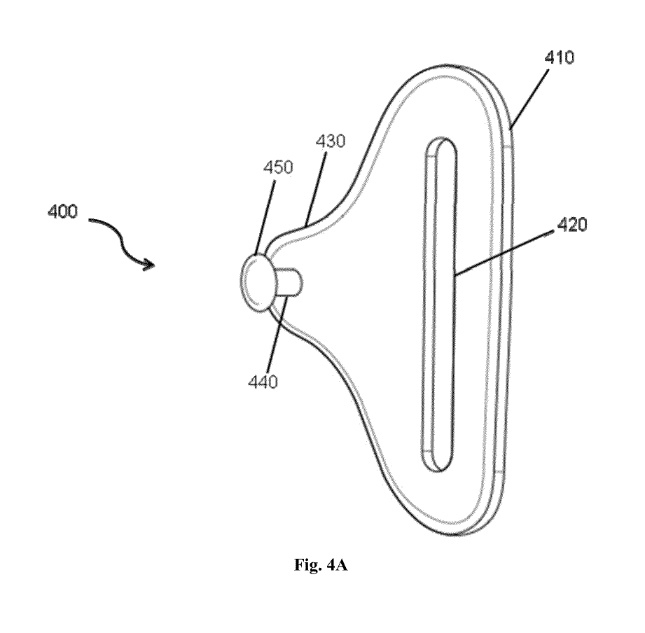

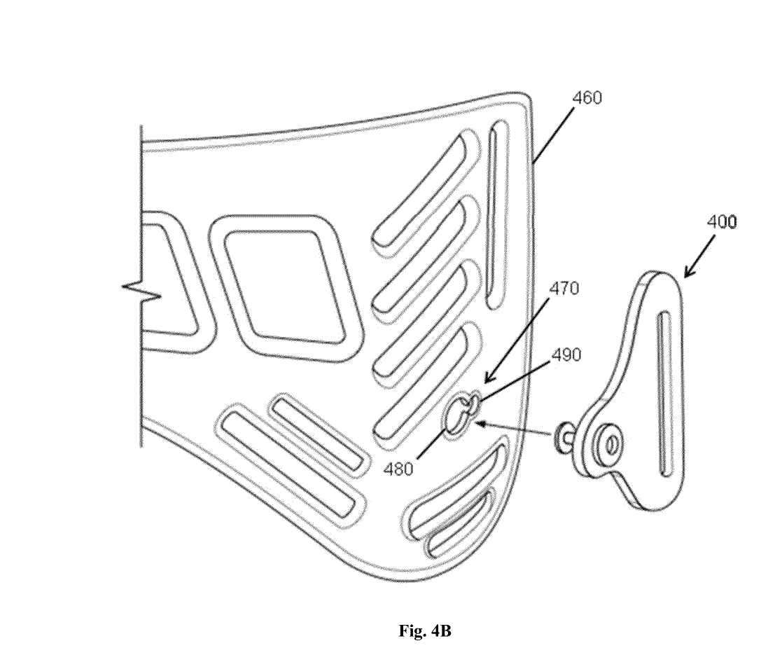

As noted above, a brace of the inventive concept can be easily donned and doffed while seated, which is particularly advantageous for person with reduce mobility and/or poor balance. Braces of the inventive concept can also include additional features that simplify application and adjustment of the device. For example straps of the brace should be fixed to or guided along the various brace components in such a manner as to allow for free movement of the braced limb, while still permitting sufficient fixation to allow for the generation of force while remaining secure. At the same time, accommodation should be made for the possibility of reduced strength, vision, and dexterity among osteoarthritis patients. An embodiment of one such fixating or guiding device is shown in FIGS. 4A and 4B. FIG. 4A depicts an embodiment of a “mushroom” connector 400 for use with straps of a brace of the inventive concept. The mushroom connect includes a strap receiver 410 and a slot 420, through which a portion of the strap passes. If a strap is looped through the slot 420, the mushroom connector 400 can serve to attach a terminus of the looped strap to a site on a brace of the inventive concept. Alternatively, if a strap is passed through the slot 420 so that it can slide, the mushroom connector can serve as a point from which a strap (for example, a force strap) is redirected. The mushroom connector 400 can also include a tab 430, which in turn supports a stem 440 that projects at an angle normal to the plane of the tab. The stem 440 includes a flared terminus 450 that is substantially parallel to the plane of the tab 430 and has a diameter greater than that of the stem 440. The flared terminus 450 can be any suitable shape, for example a cylinder, sphere, hemisphere, ovoid, rectangular prism, triangular prism, cube, or a combination of these. In a preferred embodiment, the flared terminus 450 is a portion of a sphere. An example of the utilization of a mushroom connector 400 is shown in FIG. 4B, which depicts a portion of a frame 460 of a brace of the inventive concept that includes a mushroom connector channel 470. The mushroom connector channel 470 has a receiving portion 480 and a retaining portion 490. The receiving portion 480 has a width that permits passage of the flared terminus of the mushroom connector 400, while the retaining portion 490 has a width that retains the flared terminus while permitting movement of the stem of the mushroom connector 400. In order to connect a strap associated with the mushroom connector 400 at this position merely needs to guide the flared terminus into the receiving portion 480 and allow it to move into the retaining portion 490. This arrangement secures the strap, while permitting it to swivel to permit free movement, acting as a swivel connector. In a preferred embodiment, a mushroom connector is used as a guiding device, providing an inflecting position along a force strap, such that a portion of the force strap moves through the slot 420 when the brace is in use.

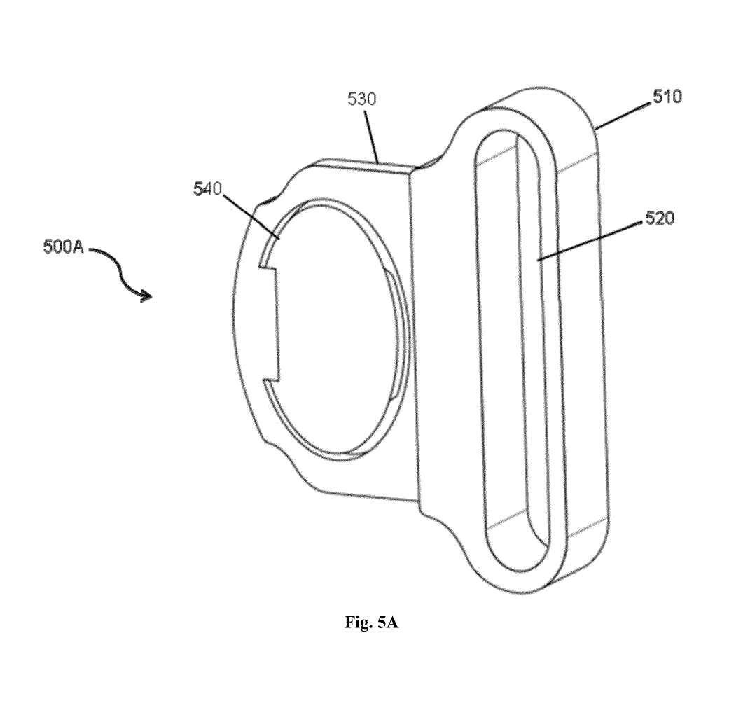

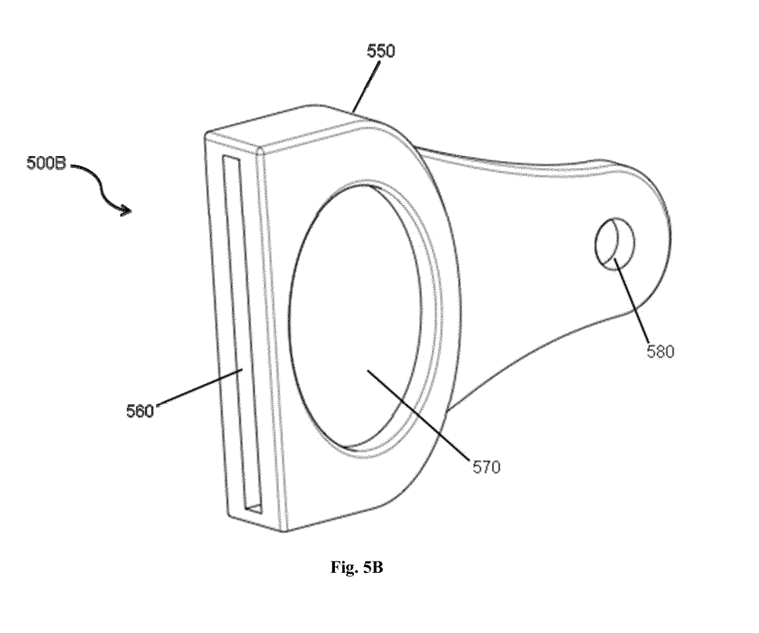

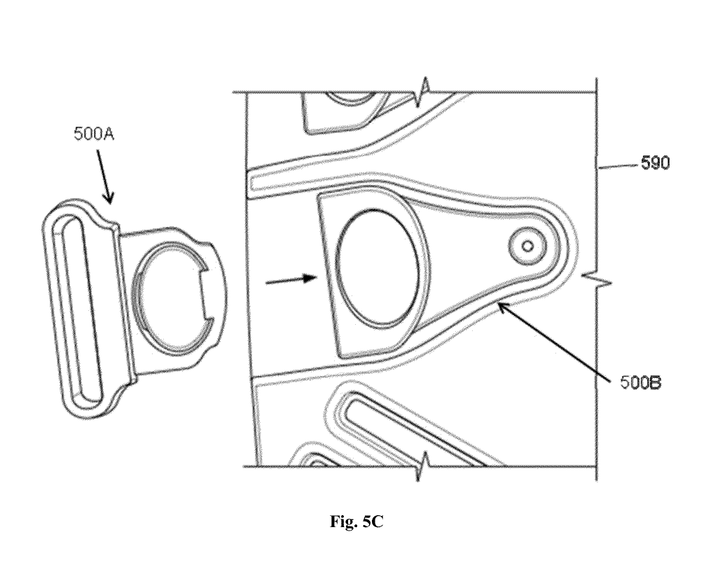

Another fixating or guiding device that is useful with braces of the inventive concept is the snap connector shown in FIGS. 5A to 5C. FIG. 5A depicts a male portion of a snap connector 500A. The male portion of the snap connector 500A includes a strap receiver 510 and a slot 520, through which a portion of the strap passes. If a strap is looped through the slot 520, a snap connector can serve to attach a terminus of the looped strap to a site on a brace of the inventive concept. Alternatively, if a strap is passed through the slot 520 so that it can slide, the snap connector can serve as a point from which a strap (for example, a force strap) is redirected. The male portion of the snap connector 500A also includes a tab 530 that supports a projecting catch 540. The projecting catch 540 extends outward from the tab 530, and is constructed, at least in part, of a suitably pliant or flexible material that permits alignment of the projecting catch 540 with the plane of the tab 530. FIG. 5B depicts a female portion of a snap connector 500B. The female portion of the snap connector 500B includes a connector body 550, which includes an interior space that accommodates the tab of the male portion of the snap connector. The connector body 550 includes a channel 560 that permits insertion of the tab of the male portion of the snap connector and an aperture 570, into which the projecting catch of the male portion of the snap connector projects when the device is assembled. The female portion of the snap connector 500B also includes an attachment point 580 that facilitates attachment to a portion of a brace of the inventive concept. Assembly of the snap connector (and therefore fixation of attached straps) is shown in FIG. 5C, which depicts a female portion of a snap connector 500B attached to a portion of a brace of the inventive concept, and a male portion of the snap connector 500A positioned for insertion. The attachment of the snap connector to a frame of the brace can be arranged to permit movement of the connector while securing the strap, permitting it to act as a swivel connector. For example, a snap connector or a portion thereof can be affixed to a frame using a rivet, bolt, screw, or similar projection placed through an aperture in the material of the snap connector so as to permit rotational movement. Alternatively, the snap connector or a portion thereof can incorporate a flexible tether that is affixed to a frame of the brace. Utilizing such a snap connector, a user can attach or guide straps used with a brace of the inventive concept (for example, force straps or stabilizing straps) by simply guiding the male portion of the snap connector into the channel of the female portion of the snap connector until the projecting catch engages the aperture. The attachment can be easily reversed by pressing against the projecting catch and pulling on the attached strap. In a preferred embodiment, a snap connector is used to affix a terminus of a strap to a frame of the brace.

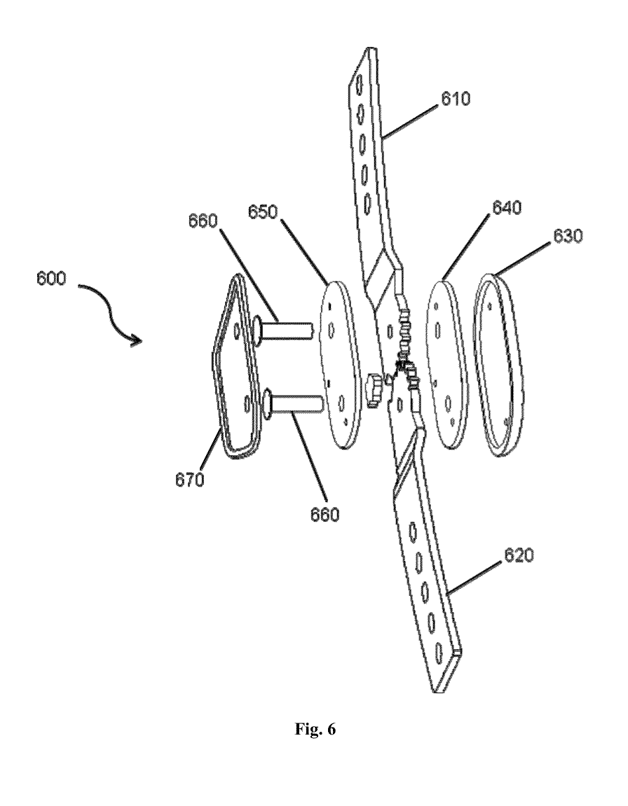

As noted above, a brace of the inventive concept can include a hinge that serves to link the frames of the brace and to mimic the natural motion of the braced joint. An example of such a hinge is shown in FIG. 6. The hinge 600 includes a first arm 610 and a second arm 620 that are attached to different frames of the brace. For example, first arm 610 can be attached to an upper frame of the brace while the second arm 620 is attached to the lower frame. As shown, such arms can have a number of holes or other attachment points that facilitate proper fitting of the hinge to the brace and allow a single model of arm to be used across a wide variety of braces. The hinge 600 can also include a cover 630, which can help protect the interior mechanism of the hinge 600, and a first plate 640 and a second plate 650 that help support and stabilize the hinge 600 against lateral stress. In some embodiments the hinge 600 can include one or more support pins 660, which in turn support a pad 670. The pad 670 is oriented towards the joint, and reduces pressure encountered by the joint on contact with the hinge. In some embodiments of the inventive concept the hinge 600 can be configured so that the pad 670 is positioned to apply pressure to the unaffected side of the braced joint. In this way the hinge 600 can act as a fulcrum, applying additional abstracting force to the joint that complements the forces applied by the upper and lower frames. Such embodiments have particular utility in more advanced osteoarthritis, where additional force is needed to properly align the joint.

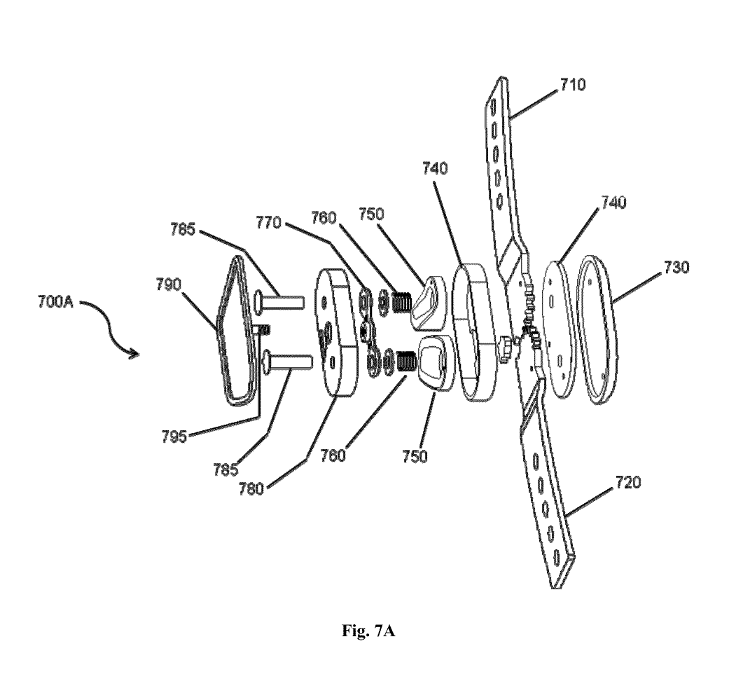

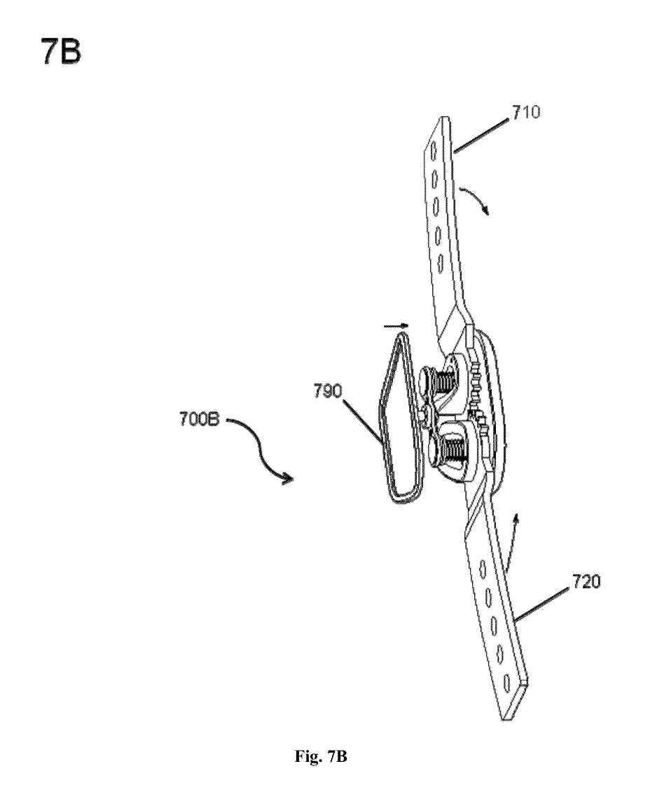

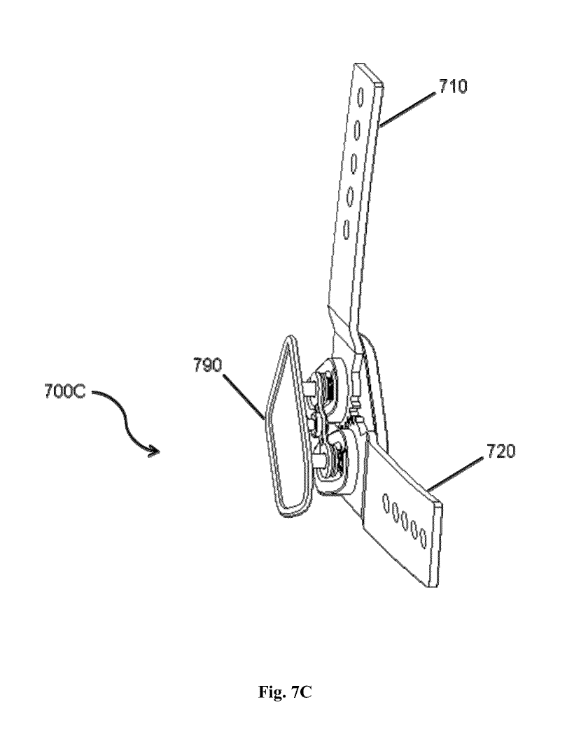

In an alternative embodiment, a brace of the inventive concept can include a ramping hinge as shown in FIGS. 7A to 7C. As shown in an exploded view in FIG. 7A, the ramping hinge 700 includes a first arm 710 and a second arm 720, which serve to link the frames of the brace. The ramping hinge 700 can also include a cover 730, that serves to protect the interior mechanism of the hinge, and a support plate 740, which aligns and supports the first arm 710 and second arm 720 against lateral stress. A casing 750 serves to enclose the ramps 750, which engage the pad support 770. The pad support 770 is impelled towards the tops of guide pins 785 by springs 760, and supports the pad 790 via a pad guide 795.

FIGS. 7B and 7C show a ramping hinge in extended and flexed configuration, respectively. As shown in FIG. 7B, with the arms 710, 720 of the ramping hinge 700B lying along a straight line (i.e. the limb straight an in weight-bearing position) the pad 790 is displaced away from the body of the ramping hinge 700B my movement of the pad support to the top portion of the ramps. This directs the pad 790 towards the unaffected side of the braced joint. FIG. 7C shows the ramping hinge 700C with the arms 710, 720 at an angle relative to one another (i.e. when the limb is flexed and not weight bearing). This permits the pad support to move along the guide pins, towards the body of the ramping hinge, dynamically relieving stress from the braced joint at times when it is not needed.

Still another embodiment of the inventive concept is a brace (i.e. a dual hinge brace) for relieving stress from a joint affected by osteoarthritis, where the brace includes an upper and a lower frame connected through an Instantaneous Center of Rotation (ICR) hinge mechanism and a push-pull hinge. The push-pull hinges moves the frames through a prescribed arc as the brace flexes, applying an abstracting force to the affected joint, which the ICR hinge mimics the natural movements of the joint and provides support. Such a brace can include additional hinges, for example one or more angulation hinges, that provide an additional, constant abstracting force. Use of such angulation hinges can be helpful in more advanced disease. In such an embodiment, the frames can be constructed of rigid, lightweight materials. Suitable materials include aluminum, stainless steel, titanium, graphite composites, and combinations of these.

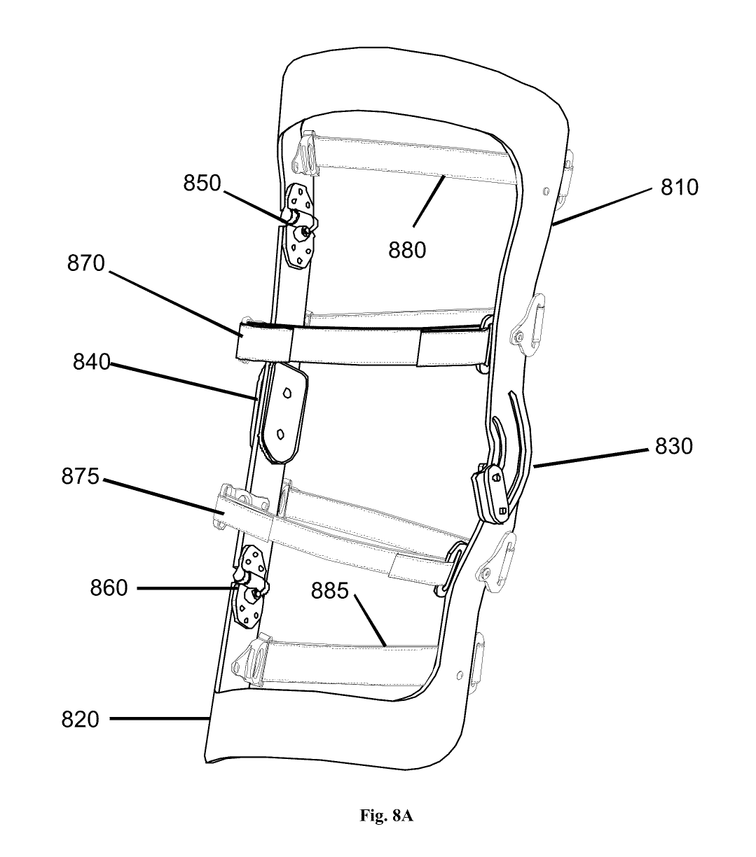

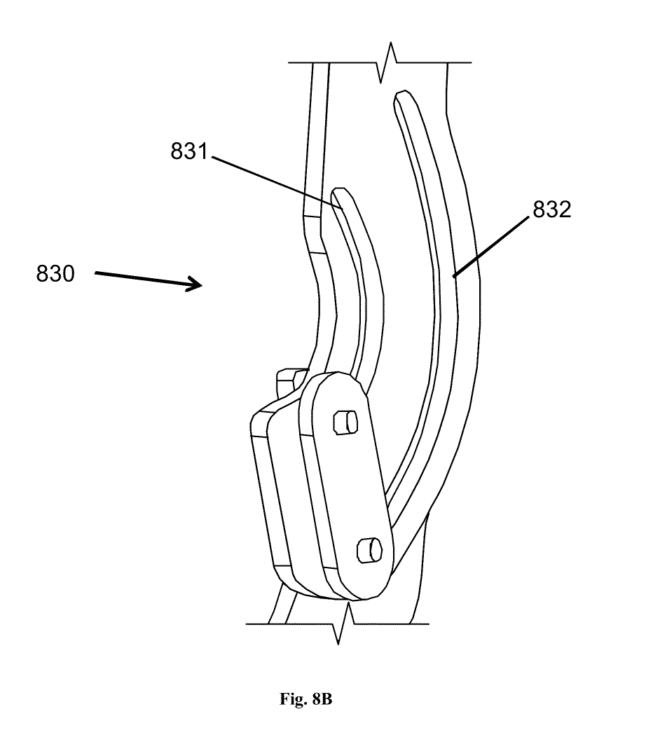

An example of such a dual hinge brace is shown in FIG. 8A. FIG. 8A depicts a dual hinge brace 800 suitable for use with a knee, and includes an upper frame 810 that is joined to a lower frame 820 by an push-pull hinge 830 on one side and an ICR hinge 840 and angulation hinges 850, 860 on the other. The brace is stabilized on the leg with upper 870 and lower 875 circumferential straps, and can be further stabilized using upper 880 and lower 885 support straps.

In such a brace the angulation hinges 850, 860 are set to provide a fixed angle between the upper frame 810 and the lower frame 820. In some embodiments of the inventive concept the angulation hinge can be placed on just one frame (typically the femoral or upper frame); in other embodiments of the inventive concept an angulation hinge may be placed on both femoral (upper) and tibial (lower) frames to provide increased unloading force. The mechanism of such an angulation hinge can utilize a worm gear principle to move the frames. In such an embodiment the movable piece of the mechanism may be attached to the frame through which it is desirable to apply the unloading force. This arrangement permits simple adjustment of an angulation hinge, for example using an allen screw incorporated into the angulation hinge mechanism, thereby setting the angle of the frame and the unloading force. In embodiments in which a second hinge is attached to the opposing frame, similar steps are followed to set the desired angle. The force thus applied will remain essentially constant throughout the range of motion of the brace, and directed to provide an unloading force to the affected joint. It should be therefore be appreciated that, while shown with two angulation hinges 850, 860, a single hinge can also be utilized. In practice, the constant application of force sufficient to unload the joint when under full weight can cause discomfort to the wearer. In order to address this issue, the brace 800 also incorporates a push-pill hinge 830.

This push-pull hinge changes the angle between the upper frame 810 and the lower frame 820 as the brace moves in and out of flexion. The effect of such a push-pull hinge 830 can be directed primarily towards one of the frames, for example the upper frame 810. In such an embodiment, if the push-pull hinge is on the lateral side, the upper frame will move towards the lateral side when the brace goes into flexion. The push-pull hinge 830 is shown in greater detail in FIG. 8B. The degree and/or distance of such a lateral (or horizontal) shift is directly related to the diameter of the arcs 831, 832 of the push-pull hinge 830, and can be controlled by using different sizes of arcs on the hinge. In some embodiments of the inventive concept, a push-pull hinge may have an arc and/or a set of arcs of essentially identical radii. In other embodiments of the inventive concept a push-pull hinge may have multiple arcs and/or sets of arcs of different radii. In still other embodiments of the inventive concept, the two arcs of the push-pull system shown in FIG. 8B can be replaced with a single arc, where a slot in one of the frames may be utilized to perform the function of the second arc. The brace also includes an ICR hinge 840, which is designed to mimic the natural movements of the knee. The difference in rotation between the ICR hinge 840 and the push pull-hinge 830 drives the change in angle of the brace as it moves through flexion.

To achieve the desired results in relief of a joint affected by OA, the frame is moved towards the direction of unloading using the angulation mechanism. The amplitude of this movement can be set by a physician, orthotist, physical therapist, or other medical professional. Alternatively, the amplitude of the movement can be user defined. For example, if the ICR hinge of a knee brace is on the medial side the user may angle the frame further in the medial direction (towards the center of the body). The push-pull hinge may then be installed on the opposite side. When the brace is in full extension there is maximum unloading as the rigid frame pushes the femoral area medially (if there is a second angulation hinge on the lower frame, there will be additional force created by the lower frame's setting as defined by its angulation hinge). Once the brace starts moving in flexion, the push-pull hinge forces the frame to move laterally, providing an unloading force to the leg.

It should be appreciated that both the angulation hinge and the push-pull hinge may be placed on the same side of the brace and still provide effective dynamic unloading. For ease of assembly, however, it may be desirable to have these two mechanisms on the opposite sides.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A brace for applying an unloading force to a joint, comprising;
a first frame having a first aspect and a second aspect;
a second frame having a first aspect and a second aspect;
a hinge, wherein the hinge is interposed between the first frame and the second frame;
and,
a first force strap comprising a first terminus, a second terminus, and a first intermediate segment interposed between the first terminus and the second terminus;
wherein the first terminus is affixed to the first aspect of the first frame and the second terminus is affixed to the first aspect of the second frame, and wherein the first intermediate segment strap is slidably engaged with the second aspect of the first frame.

2. The brace of claim 1, further comprising a second force strap, the second force strap comprising a third terminus, a fourth terminus, and a second intermediate segment interposed between the third terminus and the fourth terminus, wherein the third terminus is affixed to the first aspect of the second frame and the fourth terminus is affixed to the first aspect of the first frame, and wherein the second intermediate segment slidably engaged with the second aspect of the second frame.

3. The brace of claim 1, wherein the first force strap is inelastic.

4. The brace of claim 1, wherein the first frame further comprises a first support strap and the second frame further comprises a second support strap.

5. The brace of claim 1, wherein the hinge is configured to provide an angulation between the first frame and the second frame.

6. The brace of claim 1, wherein the first frame and the second frame comprise a pliant material.

7. The brace of claim 6, wherein the pliant material is selected from the group consisting of one or more of a polyamide, polyurethane, polypropylene, crosslinked polypropylene, polyethylene, crosslinked polyethylene, carbon fiber, fiberglass, and resin.

8. The brace of claim 1, wherein the first strap is affixed to the first frame using a swivel connector.

9. The brace of claim 8, wherein the swivel connector is a snap connector.

10. The brace of claim 1, wherein the length of the first force strap is adjustable.

11. The brace of claim 10, wherein the first force strap includes indicia, the indicia providing guidance for adjustment of the length of the first force strap.

12. The brace of claim 5, wherein the hinge includes a ramping mechanism, and wherein the ramping mechanism is configured to modify the degree of angulation between the first frame and the second frame as the hinge pivots.

13. The brace of claim 12, wherein the ramping mechanism of the hinge is adjustable by a user.

14. The brace of claim 1, wherein alignment of the first frame and the second frame along a first axis result in an increase in angulation between the first frame and the second frame in a second axis.

15. The brace of claim 13, wherein the brace is configured to be applied to a limb.

16. The brace of claim 15 wherein the first axis is parallel to a major axis of the limb to which the brace is applied.

17. The brace of claim 16, wherein the second axis is normal to a major axis of the limb to which the brace is applied.

18. The brace of claim 17, wherein the hinge comprises a pad, and wherein the pad moves along the second axis as alignment of the first frame and second frame along the first axis changes.

19. A method for providing an unloading force to a joint, comprising:
providing a brace comprising a first frame having a first aspect and a second aspect, a second frame having a first aspect and a second aspect, and a first force strap comprising a first terminus, a second terminus, and a first intermediate segment interposed between the first terminus and the second terminus, wherein the first terminus is affixed to the first aspect of the first frame and the second terminus is affixed to the first aspect of the second frame, and wherein the first intermediate segment strap is slidably engaged with the second aspect of the first frame; and,
applying the brace to a limb comprising a joint, such that the joint is interposed between the first frame and the second frame,
wherein an angulation between the first frame and the second frame is increased when the limb is extended.

20. The method of claim 19, wherein the brace further comprises a second force strap, the second force strap comprising a third terminus, a fourth terminus, and a second intermediate segment interposed between the third terminus and the fourth terminus, wherein the third terminus is affixed to the first aspect of the second frame and the fourth terminus is affixed to the first aspect of the first frame, and wherein the second intermediate segment slidably engaged with the second aspect of the second frame.

21. A method of claim 19, further comprising the step of modifying the angulation between the first frame and the second frame as the limb is extended by adjusting the length of the first force strap.

22. A brace for applying an unloading force to a joint, comprising;
a first frame;
a second frame;
a push-pull hinge, wherein the push-pull hinge is interposed between and connected to the first frame and the second frame; and, an internal center of rotation hinge, wherein the internal center of rotation hinge is interposed between and connected to the first frame and the second frame;
wherein the push-pull hinge is configured to alter the degree of angulation between the first frame and the second frame as joint is flexed.

23. The brace of claim 22, further comprising one or more angulation hinges.

24. The brace of claim 22, wherein the push-pull hinge and the internal center of rotation hinge are on opposite sides of the brace.
